(19) United States
(12) Patent Application Publication
(10) Pub. No.: US 2022/0096811 A1
(43) Pub. Date: Mar. 31, 2022

(54) DELIVERY SYSTEM ADAPTER

(71) Applicant: Boston Scientific Scimed, Inc., Maple Grove, MN (US)

(72) Inventors: Darren G. Curran, Galway (IE); Gerard Duignan, Galway (IE); John T. Favreau, Spencer, MA (US); James Quinn, Galway (IE); Ryan D. Lynch, Roscommon (IE)

(21) Appl. No.: 17/479,474

(22) Filed: Sep. 20, 2021

Related U.S. Application Data
(60) Provisional application No. 63/083,352, filed on Sep. 25, 2020.

(51) Int. Cl. A61M 39/10; A61M 39/02; A61M 39/24

(52) U.S. Cl. CPC A61M 39/1011; A61M 39/0247; A61M 2039/1033; A61M 2039/1077; A61M 2039/0279; A61M 39/24

(57) ABSTRACT

The present disclosure relates generally to the field of medical devices. In particular, the present disclosure relates to adapting medical devices for use of a distal portion of a medical device within a restricted anatomical area. In one example, a coupling device may comprise first and second ends. A first end may be configured to couple to a locking member of a medical device delivery system. The first end may comprise a lumen extending therethrough. The second end may comprise a lumen continuous with the lumen of the first end. The second end may be configured to couple to a channel opening of an endoscope. Other embodiments are contemplated.

### PRIORITY

The present application is a non-provisional of, and claims the benefit of priority under 35 U.S.C. § 119 to, U.S. Provisional Application Ser. No. 63/083,352, filed Sep. 25, 2020, the disclosures of which is herein incorporated herein by reference in its entirety for all purposes.

### FIELD

The present disclosure relates generally to the field of medical devices. In particular, the present disclosure relates to medical device delivery systems.

### BACKGROUND

Endoscopic procedures can be less invasive than traditional procedures. However, access to target tissue may be limited by the constraints of the particular instruments used. For example, proper positioning, use, and/or deployment of a medical device (e.g., stent, needle, cutting element, etc.) within an anatomical area (e.g., body lumen, passage, vessel, duct, etc.) may require the use of an endoscope with a working channel length and/or a tip configuration appropriate to facilitate access to the tissue.

It is with these considerations in mind that the improvements of the present disclosure may be useful.

### SUMMARY

In one aspect, a system may comprise a locking member of a medical device delivery system and a coupling device. The coupling device may comprise a first end comprising a portion with a threaded outer surface configured to couple to the locking member. The first end of the coupling device may further comprise a lumen extending therethrough. The coupling device may include a second end comprising a lumen axially continuous with the lumen of the first end. The second end of the coupling device may be configured to couple to an outlet of an endoscope channel.

In the described and other aspects of the present disclosure, the first end of the coupling device may comprise a Luer lock fitting. The second end of the coupling device may correspond to a larger inner diameter of the lumen than the first end. The lumen at the second end of the coupling device may comprise a first portion with a first inner diameter and a second portion with a second inner diameter. The second inner diameter may be larger than the first inner diameter. The second portion may be configured to fit over a ridge of the outlet of the endoscope channel. The system may comprise a cap coupled to the first end of the coupling device. The cap may comprise a valve. The coupling device may further comprise a middle portion between the first end and the second end. An external surface of the middle portion may comprise a surface variation. The coupling device may be further configured to couple to the locking member to create a fluid-tight fit. The lumen of the coupling device may comprise a unidirectional valve. An inner diameter of at least part of the lumen of the coupling device may be included in a range of 5-12 Fr. The second end of the coupling device may comprise a quick disconnect fitting. The first end of the coupling device, when coupled to the locking member, may be configured to prevent free rotation of the coupling device with respect to the locking member, the second end of the coupling device, when coupled to a channel of an endoscope, may be configured to prevent free rotation of the coupling device with respect to the channel, or both. The lumen of the second end of the coupling device may be configured to receive an outlet of an endoscope channel in a fluid-tight fit. When the first end of the coupling device is coupled to the locking member and the second end of the coupling device is coupled to an outlet of an endoscope channel, the lumen of the first end of the coupling device may be configured to pass a delivery sheath of the medical device delivery system into the endoscope channel. The second end of the coupling device may be configured to couple to an outlet of an endoscope channel via a press fit, a turn lock connection, or a clip fit.

In another aspect, a system for connecting a medical device to an endoscope may comprise a locking member of the medical device and a coupling device. The coupling device may comprise a first end comprising threading along an outer surface thereof configured to be coupled with the locking member, wherein the locking member may be disposed around a sheath of the medical device. The coupling device may comprise a second end configured to be coupled with an outlet of a channel of an endoscope. In some embodiments, the coupling device may comprise a lumen extending longitudinally along a full length of the first end and the second end. The second end of the coupling device may be configured to couple to the outlet via insertion of the outlet into the lumen.

In the described and other aspects of the present disclosure, the coupling device may be configured to couple to the locking member to create a fluid-tight fit, to the outlet to create a fluid-tight fit, or both. The lumen of the coupling device may comprise a unidirectional valve. An inner diameter of at least part of the lumen of the coupling device may be included in a range of 5-12 Fr. The first end of the coupling device, when coupled to the locking member, may be configured to prevent free rotation of the coupling device with respect to the locking member, the second end of the coupling device, when coupled to a channel of an endoscope, is configured to prevent free rotation of the coupling device with respect to the channel, or both. When the first end of the coupling device is coupled to the locking member and the second end of the coupling device is coupled to an outlet of an endoscope channel, the lumen of the first end of the coupling device may be configured to pass a delivery sheath of the medical device delivery system into the endoscope channel. The second end of the coupling device may be configured to couple to an outlet of an endoscope channel via a press fit, a turn lock connection, quick disconnect fitting, or a clip fit.

In yet another aspect, a system for coupling a delivery system to an endoscope may comprise a locking member of a delivery system and an adapter. The adapter may comprise a first end configured to, when inserted into a lumen of a locking member of the delivery system, secure the adapter to the locking member. The adapter may comprise a second end configured to clip over a ridge of a channel opening of the endo scope. When the second end is clipped over the ridge of the channel opening, the first end may form a lumen providing access through the adapter to the channel opening.

### BRIEF DESCRIPTION OF THE DRAWINGS

Non-limiting embodiments of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment shown where illustration is not necessary to allow those of ordinary skill in the art to understand the disclosure. In the figures:

FIGS. 1A-B respectively illustrate an exemplary medical device delivery system and an exemplary locking member of a medical device delivery system;

FIG. 2 illustrates an exemplary endoscope;

FIGS. 3A-3B respectively illustrate a side view and a perspective view of an adapter according to one embodiment of the present disclosure;

FIG. 4 illustrates a system for delivering a medical device according to one embodiment of the present disclosure;

FIG. 5 illustrates a cross-sectional view of components for delivering a medical device according to one embodiment of the present disclosure;

FIGS. 6A-6B illustrate cross-sectional views of an adapter according to one embodiment of the present disclosure.

FIGS. 7A-7B illustrate additional cross-sectional views of adapters according to several embodiments of the present disclosure.

FIGS. 8A-8B respectively illustrate cross-sectional and perspective views of an additional adapter according to one embodiment of the present disclosure.

FIGS. 9A-9D illustrate cross-sectional and perspective views of additional adapters according to various embodiments of the present disclosure.

FIGS. 10A-10B respectively illustrate cross-sectional and perspective views of an additional adapter according to one embodiment of the present disclosure.

FIGS. 11A-11B respectively illustrate cross-sectional and perspective views of an additional adapter according to one embodiment of the present disclosure.

FIG. 12 illustrates an additional medical device delivery system, which may be used in various methods of the present disclosure.

### DETAILED DESCRIPTION

The present disclosure is not limited to the particular embodiments described herein. The terminology used herein is for the purpose of describing particular embodiments only and is not intended to be limiting beyond the scope of the appended claims. Unless otherwise defined, all technical terms used herein have the same meaning as commonly understood by one of ordinary skill in the art to which the disclosure belongs.

Although embodiments of the present disclosure may be described with specific reference to stent delivery systems for the sake of brevity, it should be appreciated that such delivery systems may be used to deliver a variety of medical devices (e.g., aneurysm coils, needles, cutting elements, biopsy tools, other implants, and the like) into a variety of restricted anatomical areas, including, but not limited to, body lumens (e.g., stomach, pancreato-biliary tree, colon, duodenum, jejunum, uterus, etc.), passages, vessels and/or ducts, to address a variety of conditions. For example, a delivery system may be a biopsy device, a stent delivery device, a fine needle aspiration (FNA) device, or other medical device.

As used herein, the singular forms “a,” “an,” and “the” are intended to include the plural forms as well, unless the context clearly indicates otherwise. It will be further understood that the terms “comprises” and/or “comprising,” or “includes” and/or “including” when used herein, specify the presence of stated features, regions, steps, elements and/or components, but do not preclude the presence or addition of one or more other features, regions, integers, steps, operations, elements, components and/or groups thereof.

As used herein, the term “distal” refers to the end farthest away from the medical professional or physician when introducing a device into a patient, while the term “proximal” refers to the end closest to the medical professional or physician when introducing a device into a patient.

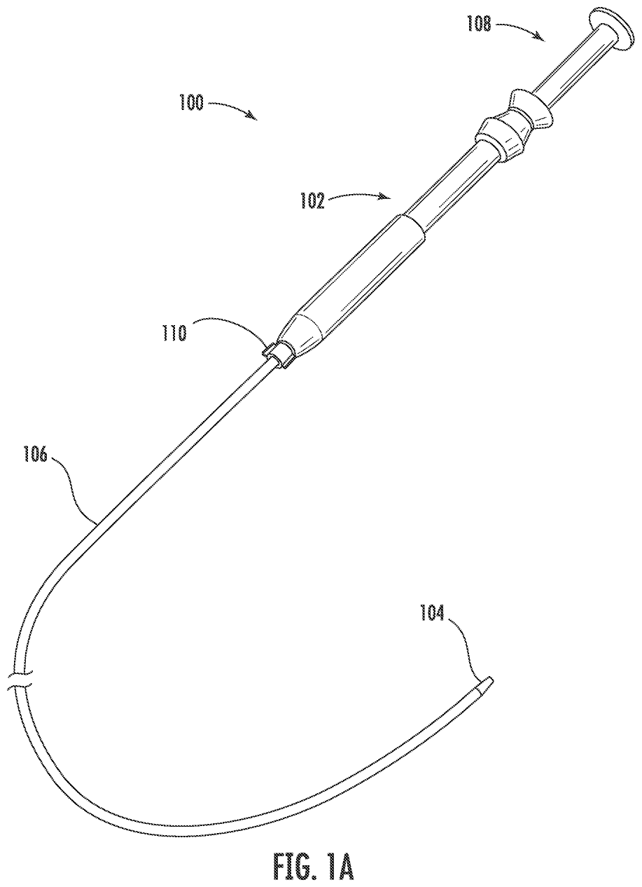

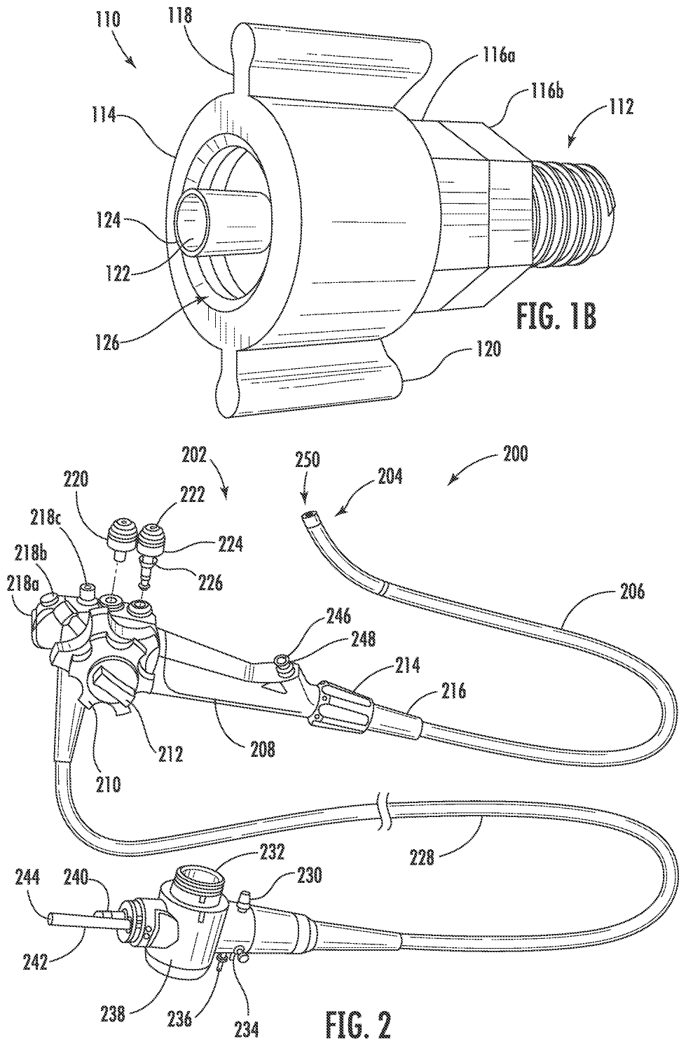

Referring to FIGS. 1A-B, a delivery system 100 may be used to deliver a medical device to a target tissue. For example, the medical device may be a stent, a needle, or the like. The delivery system 100 may comprise a handle 102, a tip 104, and an elongate body, delivery sheath, or shaft such as shaft 106, extending therebetween. An actuator 108 may be used to advance the shaft 106 and/or a medical device coupled thereto. The delivery system 100 may be typically used in coordination with an endoscope, a non-limiting example of which is an endoscopic ultrasound (EUS) scope (not shown). A locking member, such as locking member 110, may be used to fix the delivery system's position with respect to an endoscope.

The locking member 110 is illustrated in greater detail in FIG. 1B. A first end 112 of the locking member 110 may be configured to couple to a handle of a delivery system so that the locking member 110 is fixed in position with respect to the handle 102. For example, threading disposed on an external surface of first end 112 may couple with corresponding threading on an internal surface of a lumen at an end of the handle 102.

A second end 114 of the locking member 110 may be rotationally fixed to the first end 112 (e.g., to form a single piece), or the second end may be able to rotate with respect to the first end. The second end 114 may include at least one projection or surface texture along an external surface thereof. The illustrated embodiment shows projections 118 and 120, which may be used to allow a user to grasp the second end 114. Surface features such as projections 118 and 120 may be useful to rotate the second end about a longitudinal axis therethrough.

The locking member 110 may comprise a lumen 122 extending therethrough. The locking member lumen 122 may extend through an entire length of the locking member. In some embodiments, the locking member 110 may comprise a discrete cylindrical opening 124, which may define the lumen 122 within the locking member 110. The locking member lumen 122 may have an internal diameter equal or greater to an external diameter of a shaft 106 of a delivery system 100 to be received therethrough such that the shaft may extend through the locking member.

The second end 114 of the locking member 110 may be configured to couple to a medical device such as an EUS (not shown), as described above. In many embodiments, a bore 126 of the second end 114 may have threading on an internal surface thereof. For example, bore 126 may have threading that corresponds to the external threading of a Luer lock fitting. The second end 114 may also or alternatively be configured to couple to a port of an endoscope channel having corresponding threading so that a shaft of a delivery system may extend through the locking member 110, through the port, and into the channel. Coupling the first end 112 of the locking member 110 to the handle 102 of the delivery system 100, and coupling the second end 114 of the locking member 110 to an endoscope can fix the delivery system 100 to the endoscope, increasing stability of the delivery system 100 to facilitate use, manipulation, and/or delivery of a medical device to target tissue (not shown).

As will be appreciated, various endoscopes may not be configured to couple directly to a particular delivery system 100. For example, the endoscope and delivery system 100 may not have compatible threads to allow the two elements to be coupled together. As such, with prior arrangements, in order to use a desire delivery system 100 for a particular procedure, a physician may be required to use an endoscope which, while compatible with the delivery system 100, may be less well suited to the particular procedure. For example, various endoscopes may comprise differing working channel lengths, different catheter diameters, different tip configurations, or the like. Use of an endoscope that is not ideally suited for a particular procedure may limit accessibility of a target tissue, may not permit the same visibility of a target tissue or may result in other limitations.

It is with considerations such as these in mind that the improvements of the present disclosure may be useful. For example, adapters, couplers, connecting members, or the like as described herein are disclosed for allowing a medical device delivery system, such as the delivery system 100 described in FIGS. 1A-B, to be coupled to an endoscope 200 (see FIG. 2), where the delivery system and the endoscope may not be configured to allow direct coupling to each other. The adapter (e.g., adapter 300) may be couplable to the delivery system 100 to prevent free rotation of the adapter 300 with respect to the locking member 110. In some embodiments the adapter 300 may be couplable to a channel 250 of endoscope 200 to prevent free rotation of the adapter with respect to the channel, thereby preventing and/or limiting undesired movement of a coupled delivery system with respect to a coupled endoscope channel. Accordingly, improvements of the present disclosure may include enabling use of medical device delivery systems with a greater variety of endoscopes, improved access and/or visibility of target tissues in procedures, improved security of positioning of medical device delivery systems during a procedure, and/or other benefits.

FIG. 2 illustrates an exemplary endoscope which may be used with a medical device delivery system 100 as described with respect to FIGS. 1A-B. It will be understood, however, that embodiments of the present disclosure may be configured for use with a variety of endoscopes and that embodiments are not limited to the specific example of FIG. 2.

Endoscope 200 may be a gastroscope, bronchoscope, cystoscope, duodenoscope, enteroscope, colonoscope, dual-channel gastroscope, or other form of endoscope. The endoscope 200 may comprise a handle 202, a tip 204, and a shaft 206 (or elongate body, or catheter) extending therebetween, where the tip 204 and at least a portion of the shaft 206 are configured to be introduced into a body cavity during an endoscopic procedure. The handle 202 may comprise a control section 208, which may include various controls for manipulating and/or securing a position of the tip 204 and/or shaft 206 from the handle 202. For example, an angulation knob 210, angulation lock 212, and/or insertion tube stiffness control 214 may be useful for manipulating a tip 204 and/or shaft 206. A boot 216 may provide support to the shaft 206 extending from the handle 202. At least one remote switch 218a-c may be useful for controlling a tool positioned at the tip 204. A suction valve 220 and/or air/water valve 224, for example, including a vent hole 222 and an anti-reflux valve 226 may be useful for regulating fluid flow through the tip 204. A universal cord 228 may extend to at least one connector component, for example, comprising a suction connector 230, a video processor connection 232, an air supply connector 234, a water supply connector 236, a light source connector 238, an air pipe 240, and/or a light guide 242, which may comprise a quartz lens 244. It will be understood that an endoscope may comprise various components and/or connections in order to support various endoscopic procedures.

The endoscope 200 may comprise at least one lumen, or channel 250 (collectively referred to herein, for brevity, as “channel”), extending longitudinally through a shaft and a tip. The channel 250 may be accessible from a handle 202 end of endoscope 200, for example, via a port or outlet such as channel opening 246. Various embodiments may include an increased diameter flange, or ridge 248 (collectively referred to herein, for brevity, as “ridge”) disposed around an opening of a channel for coupling to a delivery system 100 such as the one described in relation to FIG. 1A. It will be appreciated that many embodiments may not include compatible features for coupling with, for example, threading of a locking member 110 of a delivery system 100 as described with respect to bore 126 of FIG. 1B.

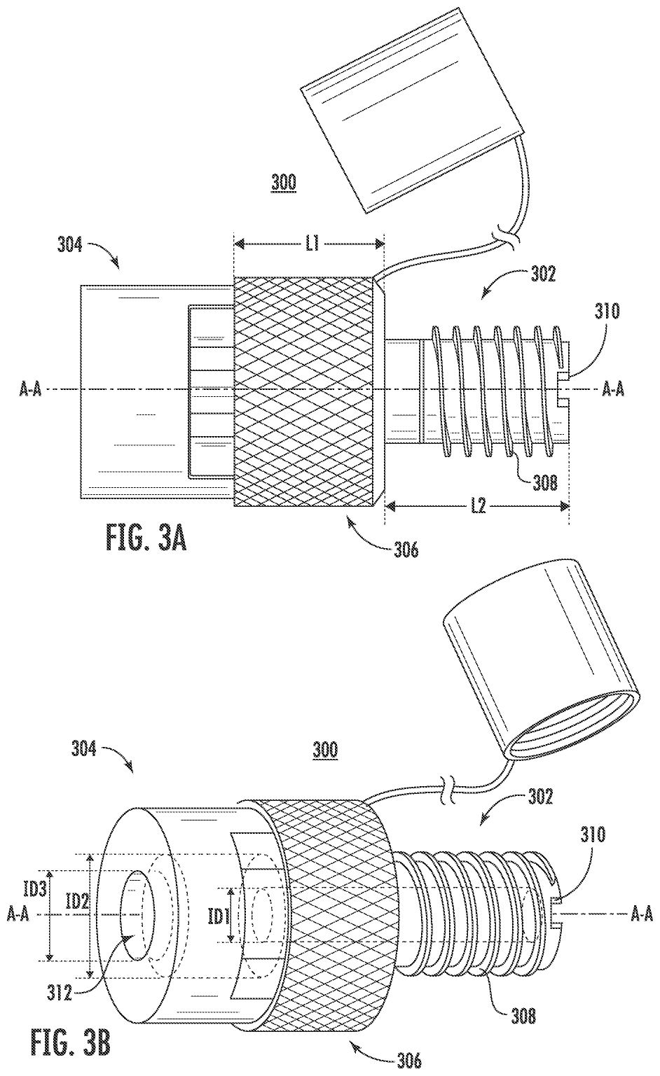

FIGS. 3A-B illustrate an exemplary adapter 300 for facilitating coupling of a delivery system 100, for example, as described with respect to FIGS. 1A-B, with endoscope 200, for example, as described with respect to FIG. 2. FIG. 3A shows a side view of adapter 300, and FIG. 3B illustrates a perspective view of adapter 300. Adapters as described herein may be made of a metal, a polymer, an elastomer, or other appropriate material, including any combinations thereof.

A first end 302 of the adapter 300 may be configured to couple to the handle 102 of the delivery system 100. A second end 304 of the adapter 300 may be configured to couple to a channel 250 of endoscope 200. In the illustrated embodiment, the first end 302 may have a smaller outer diameter than the second end 304. In some embodiments, a middle portion 306 may extend between the first and second ends 302, 304 and may have a knurled or otherwise textured outer surface for grasping by a user. In some embodiments, the middle portion 306 may alternatively or additionally comprise at least one projection, such as projection 118 or 120 described with respect to FIG. 1B. It will be understood that a middle portion 306 may comprise a length “L1”, which may correspond to a difference in working lengths between shaft 106 of delivery system 100 and working channel 250 of endoscope 200. In many embodiments, length L1 and a length of the first end 302 may sum to the difference in working lengths between shaft 106 of delivery system 100 and working channel 250 of endoscope 200. Accordingly, adapter 300 may enable use of endoscope 200 with a different working channel length than delivery system 100. As mentioned, a textured outer surface, projection, or any combination thereof may provide a grip by which one or both ends of the adapter may be rotated about a longitudinal axis thereof. In some embodiments, the second end 304 may comprise a textured external surface and/or at least one projection additionally or alternatively to the middle portion 306. Some embodiments (not shown) may comprise first and second ends directly coupled to one another without a middle portion 306 therebetween. In other embodiments, the first and second ends of the adapter may be rotatable with respect to each other.

In some embodiments, at least part of the first end 302 of the adapter 300 may include external threads 308 configured to couple with corresponding internal threads of bore 126 of the delivery system 100. In other embodiments, the first end 302 may comprise a Luer lock configured to couple with locking member 110 of delivery system 100. In further embodiments, the first end 302 may comprise one or more notches 310 or other surface features configured to interact with a corresponding surface of locking member 110 of delivery system 100 in order to provide a tight coupling of the adapter to the handle.

In many embodiments, the adapter 300 may include a lumen 312 extending therethrough along a longitudinal axis A-A of the adapter, as shown in FIG. 3B. In some embodiments the lumen 312 may have a diameter “ID1” at the first end 302 which is smaller than a diameter “ID3” at the second end 304. The lumen 312 may have a continuously increasing diameter from the first end 302 to the second end, or the lumen 312 may have a stepped configuration within the adapter 300, for example, having a step with diameter “ID2.” In many embodiments, the lumen 312 may have a diameter D1 at the first end which is larger than the outer diameter of the shaft of a medical device delivery system, for example, catheter 106 as described with respect to FIG. 1A.

In many embodiments, the lumen may have a diameter ID3 at the second end 304 which is larger than the diameter of at least part of a port of endoscope channel opening 246. For example, at least part of a lumen of end 304 may be wider than ridge 248 of endoscope 200 as described with respect to FIG. 2. The lumen of the second end 304 of the adapter 300 may couple to a port of an endoscope channel 250, as will be described in greater detail below.

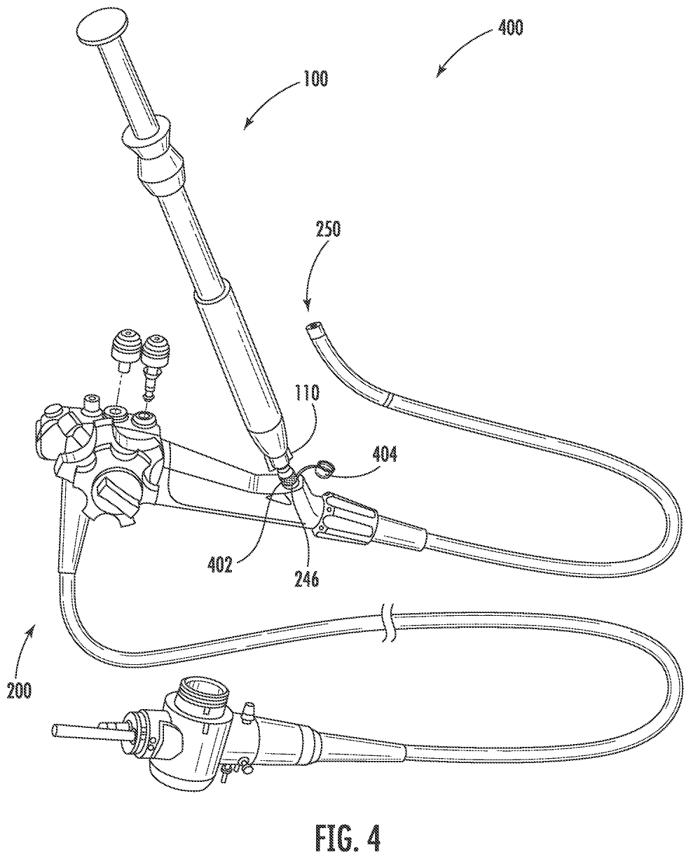

FIG. 4 illustrates an exemplary assembly of delivery system 100 and endoscope 200, according to one or more embodiments herein. The assembly 400 comprises the delivery system 100 with the locking member 110 as described with respect to FIGS. 1A-B, the endoscope 200 as described with respect to FIG. 2, and an adapter 402 coupling the endoscope channel opening to the locking member 110. The adapter 402 may have the same or similar features as adapter 300 of FIGS. 3A-B, including a first end coupled to the locking member 110, a second end coupled to an endoscope channel opening, and a lumen extending axially along the entire length of the adapter (not shown). The adapter 402 further includes a cap 404 tethered to a side of the second end of the adapter 402, which may be coupled (e.g., tightened) onto the first end of the adapter 402 when the first end is not coupled to locking member 110, thereby preventing fluid from within endoscope 200 from flowing through the channel opening (e.g., channel opening 246 as described with respect to FIG. 2) and adapter 402, It will be understood that a cap may be otherwise coupled to or unattached to an adapter in some embodiments.

When adapter 402 is used to couple delivery system 100 to endoscope 200 as described herein, a shaft 106 of the delivery system may extend through the lumen of the adapter, through the channel opening 246 of endoscope 200, and through channel 250. In many embodiments, the adapter 402 may fix a position of the delivery system 100 with respect to the endoscope 200, thus providing an operator with stability with which to manipulate or otherwise use the medical device delivery system during an endoscopic procedure. In many embodiments, the adapter 402 may form a fluid-tight fit with locking member 110 of delivery system 100 and/or with channel opening 246 of endoscope 200.

FIG. 5 illustrates a cross-section view of a system according to one or more embodiments herein. The system may include endoscope 200, adapter 502, and delivery system 100. For the sake of clarity, the system of FIG. 5 is illustrated in a partially exploded view.

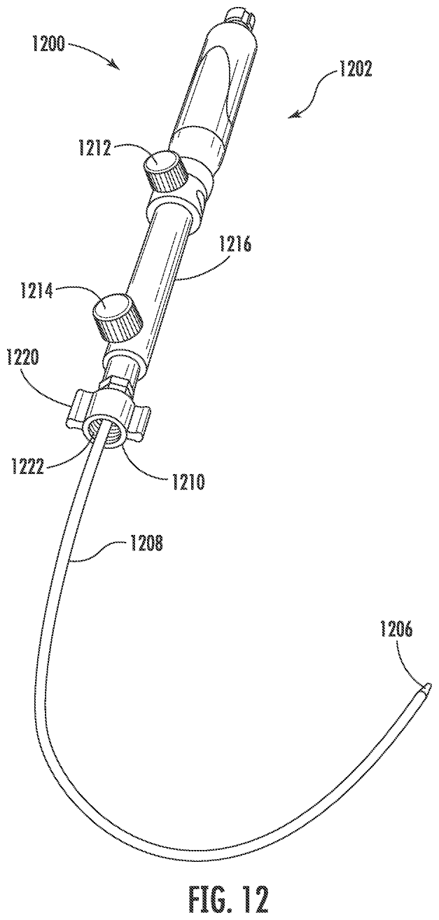

Adapter 502 may be configured to couple endoscope 200, to delivery system 100. While endoscope 200 and delivery system 100 are presented by way of example with respect to FIG. 5, it will be understood that alternative endoscopes and/or delivery systems may be used, for example, delivery device 1200 of FIG. 12. Adapter 502 may have the same or similar features as adapter 300 as described with respect to FIGS. 3A-B, including a first end coupled to the locking member 110, a second end coupled to endoscope channel opening 246, and a lumen extending axially along the entire length of the adapter.

The adapter 502 may comprise a cap 504, which may be configured to fit over an end of the adapter, and, in many cases, can provide a fluid-tight seal thereover. For example, cap 504 is configured to fit over end 512 of adapter 502. Cap 504 comprises threading 506 on an internal surface configured to interact with a corresponding threaded outer surface 520 of end 512, thereby coupling the cap to the end. It will be understood, however, that the cap 504 may additionally and/or alternatively comprise an internal surface configured to couple with an end of an adapter via a press fit or friction fit, a suction fit, a quick-release connection, and/or other type of fitting. For example, the cap 504 may comprise a smooth internal surface or an internal surface with alternative surface variations. In many embodiments, the cap 504 may be configured to couple with an end 512 of an adapter 502 via a Luer lock connection.

The cap 504 may, in some embodiments, comprise one or more valves. For example, a plurality of flaps 508a-b may form a valve at the end of cap 504. The valve may be fluid-tight when in a closed configuration, and may be configured to move to a partially or fully open configuration, for example, when pressure is applied to the flaps 508a-b, such as when a tool, a catheter, or other medical device is inserted through the valve. In many embodiments, the valve may be configured to maintain a fluid-tight fit around a tool, catheter, or other medical device inserted therethrough. The valve may move between an at least partially open and a closed configuration while coupled or uncoupled to an end of the adapter 502. For example, if adapter 502 is uncoupled from the delivery system 100, cap 504 may be coupled to end 512 of adapter 502 (not shown), and a tool may be inserted between flaps 508a-b. A fluid-tight seal may thereby be maintained about the tool. Although the valve has been described as including flaps, it will be appreciated that the valve may comprise one or more slits, petals, bristles, duckbills, or other appropriate configuration. In various embodiments the valve may be a unidirectional valve. The cap 504 may be attached to or detached from the adapter 502, for example, via a connector such as tether 510.

The adapter 502 may comprise first and/or second ends 512, 514, where the first end is configured to couple with the delivery system 100 and the second end is configured to couple with the endoscope 200. For example, adapter 502 has a first end 512 configured to couple with locking member 110 of delivery system 100, and a second end 514 configured to couple with channel opening 246 of endoscope 200.

The first end 512 may couple with the delivery system 100 via a threaded connection, a snap fit, a press fit or friction fit, a suction fit, a Luer lock connection, a quick-release connection, or other appropriate connection, including combinations thereof. For example, first end 512 of adapter 502 has an outer diameter “OD1” which may correspond to a diameter “D2” of the bore 126 of delivery system 100 so that at least a portion of the first end is receivable within the bore. In many examples, the first end 512 of adapter 502 comprises threading along at least a portion of an external surface thereof, which is configured to interact with corresponding internal threading of bore 126 of locking member 110. First end 512 may comprise a length L2, as described above with respect to FIG. 3A (not shown).

Additionally, or alternatively, the second end 514 may couple with an endo scope 200 via a threaded connection, a snap fit, a press fit or friction fit, a suction fit, a Luer lock connection, a quick-release connection, or other appropriate connection, including combinations thereof. For example, second end 514 is configured to form a snap fit over channel opening 246 and ridge 248 of endoscope 200. In some embodiments one or more internal openings within the second end 514 of the adapter 402 may be sized and configured to receive and retain connecting features of the endoscope 200 therein. For example, first internal opening portion 516 disposed within the second end 514 may have a larger inner diameter (“ID2”) than a second internal opening portion 518 having inner diameter “ID3”. The inner diameter “ID2” of the first internal opening portion 516 may correspond to an external diameter of ridge 248 of endoscope 200, while the inner diameter “ID3” of the second internal opening portion 518 may correspond to an outer diameter of the channel opening 246. Thus, the ridge 248 and channel opening 246 of endoscope 200 may be received within the first and second internal opening portions 516, 518 to couple the endoscope to the adapter. In many embodiments, the first and second ends 512, 514 of adapter 502 may comprise different connection types.

A middle portion 524 of the adapter 502 may extend between the first end 512 and the second end 514. While middle portion 524 is illustrated as having a smooth surface, it will be recognized that an external surface thereof may include one or more surface variations, for example, texturing similar to that described in relation to middle portion 306 of FIGS. 3A-B. Middle portion 524 may comprise a length L1, as described above with respect to FIG. 3A (not shown).

It will be recognized that the adapter 502 may be made from a material that facilitates connection between the endoscope 200 and/or delivery system 100. For example, the first end 514 of the adapter may be made of an elastically deformable material, such as an elastomer, polymer or the like, which may bend, stretch, or otherwise deform to allow portion 518 to slide or snap over ridge 248 of endoscope 200. As will be appreciated, an adapter comprising a rigid or semi-rigid material may hold a delivery system 100 and/or endoscope 200 more securely and thus, adapter 502 may comprise one or multiple materials based on benefits of their properties to desired functions of the adapter. For example, adapter 300, as illustrated in FIGS. 3A-B, may comprise a first end 302 and a middle portion 306 made of a metal, such as stainless steel, which may maintain a rigidity of the adapter, while the second end 304 may be made of a polymer, elastomer or the like which may deform to allow a snap fit over a flange or ridge portion of the endoscope. Embodiments are not limited in this context.

Adapter 502 may comprise a lumen 522 extending longitudinally therethrough. When the second end 514 of the adapter 502 is coupled to the endoscope 200 (e.g., snapped over ridge 248 of channel opening 246), lumen 522 may provide access from first end 512 through the adapter to the endoscope (e.g., to the channel opening). The lumen 522 may have a continuous inner diameter from the first end 512 to the second end 514 of the adapter 502. In many embodiments, the lumen 522 may be configured to receive a shaft 106, catheter, or other medical device therethrough and to direct the shaft, catheter or other medical device into a channel opening 246 of endoscope 200.

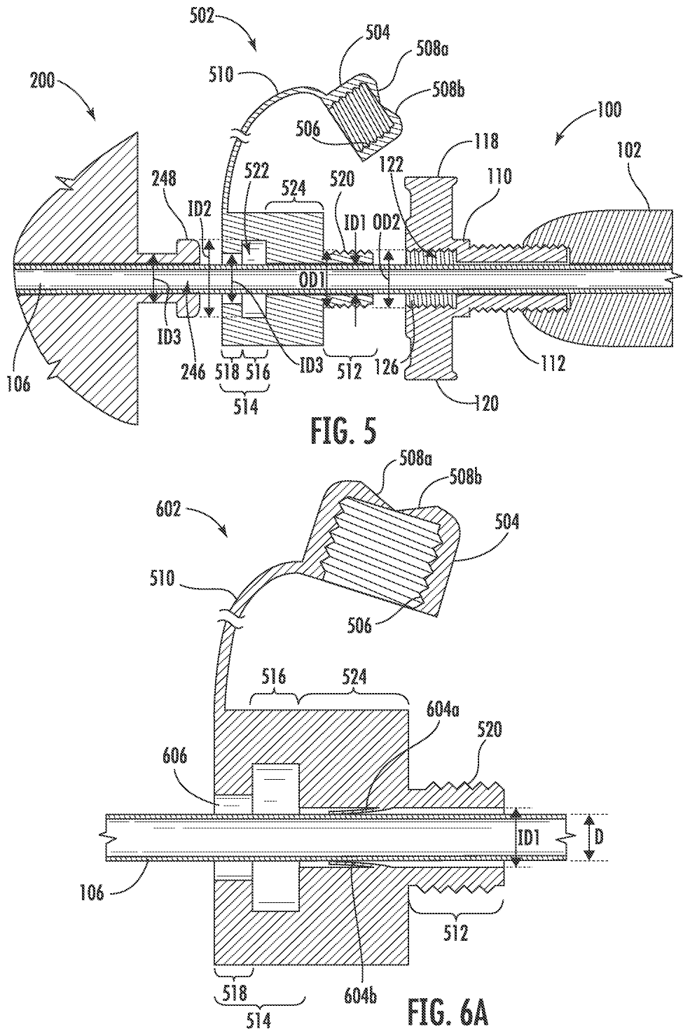

The lumen may be configured to form a fluid-tight fit with the shaft of a delivery system that is received therethrough, for example, via a friction fit. For example, adapter 602 of FIGS. 6A-B may include a lumen 606 therethrough having an inner diameter “ID1” which is larger than a diameter “D” of a delivery system shaft 106 positioned therein, as shown in FIG. 6A. The adapter 602 may include a plurality of flaps 604a-b disposed within the lumen 606 may form a valve that can form a fluid-tight fit with the shaft 106. When the shaft 106 is inserted through the lumen, the flaps 604a-b may flex to engage the outer surface of the shaft to form a fluid tight seal about the shaft, as shown in FIG. 6A. When the shaft 106 is removed, the flaps 604a-b may move to a closed configuration, as shown in FIG. 6B. The valve may be configured to move between closed and open configurations based on an application of pressure thereon, for example, by a catheter, sheath, or other medical device being inserted through the lumen 606. It will be understood that an adapter lumen 606 may incorporate any number of lumen valves, which may be the same or different type. For example, a lumen valve may comprise one or more slits, flaps, petals, bristles, duckbills, or other appropriate configuration. In some embodiments the lumen valve may be a unidirectional valve. It will be understood that any of the adapters described herein may comprise at least one lumen valve.

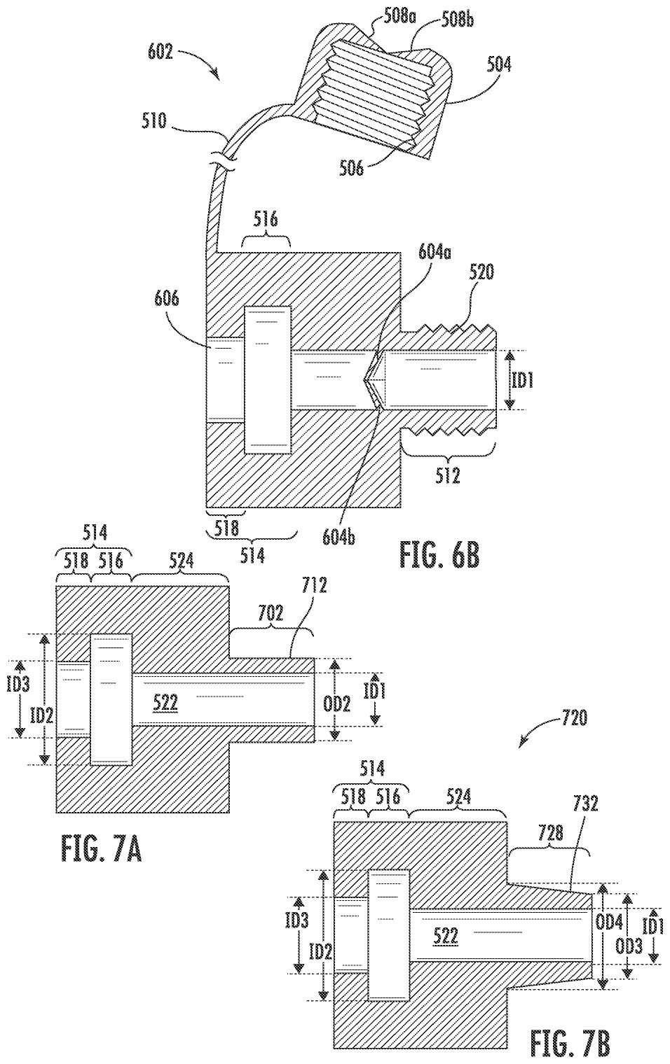

As previously noted, adapters according to the disclosure may comprise any of a variety of connection types and combinations of connection types. FIGS. 7A-B, 8A-B, 9A-C, 10A-B, and 11A-B show several examples of adapter ends configured to couple to medical devices including delivery systems and endoscopes. While, for the sake of brevity, not all possible permutations of connection types are illustrated, it will be appreciated that the end types described and/or shown may be combined in any way to form an effective adapter.

FIGS. 7A-B show cross sectional views of exemplary adapters having alternative end configurations couplable to various medical devices, such as to delivery systems with locking members having end connections that are not threaded. Adapters 700 and 720 comprise one or more components similar to those described above, for example, lumen 522 with an internal inner diameter ID1, a second end 514 with a first internal opening portion 516 comprising an inner diameter ID2, a second internal opening portion 518 comprising an inner diameter ID3, and middle portion 524. Rather than having a threaded first or second end, however, adapter 700 has a first end 702 with a smooth cylindrical surface 712 for coupling to a compatible surface of a delivery system 100. Cylindrical surface 712 may have an outer diameter “OD2” sized to create a press fit or friction fit with a lumen inner surface of a medical device, such as a locking member of a delivery system having a corresponding inner diameter (not shown). Alternatively, adapter 720 can have a first end 728 that has a tapered surface 732. It will be appreciated that tapered surface 732 may form the shape of a truncated cone. Tapered surface 732 is illustrated with a medially increasing external diameter (e.g., from outer diameter “OD4” to outer diameter “OD3”). It will be understood that tapered surface 732 may comprise different angles than that illustrated in FIG. 7B, for example, so as to provide first end 728 with a medially decreasing diameter to form a snap or clip fit (e.g., from OD3 to OD4, not shown). First end 728 may be configured to fit a corresponding surface of a medical device so as to couple the medical device to the adapter. Embodiments are not limited in this context.

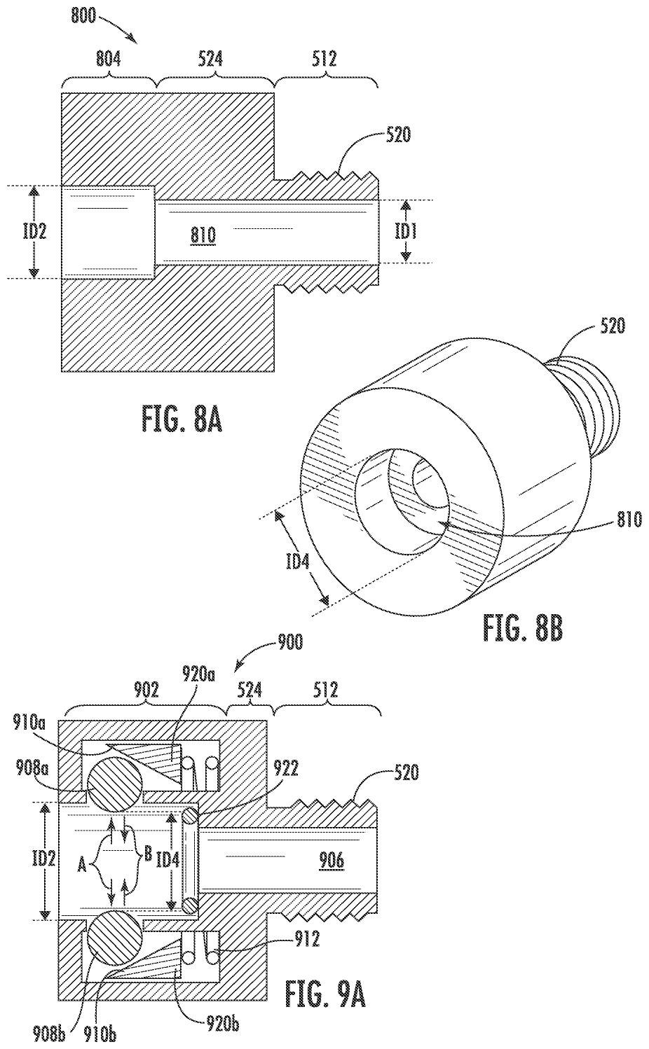

FIG. 8A shows a cross sectional view of an exemplary adapter with an alternative end configuration couplable to medical devices, such as to endoscope channel openings. FIG. 8B shows a perspective view of adapter 800. For the sake of simplicity, adapter 800 is illustrated with a middle portion 524 and an end 512, including threaded outer surface 520, as described above. It will be recognized that the adapter 800 may include a second end 804, which may comprise a constant corresponding lumen inner diameter ID2 for coupling to a compatible surface of an endoscope 200. The adapter 700 may include a lumen 810 having two discrete diameters (e.g., ID1 and ID2) as opposed to the three diameters of lumen 522 (e.g., ID1, ID2, and ID3). The constant diameter ID2 of the second end 804 of the adapter 800 may be sized and configured to couple to a component of a medical device to form a press fit or friction fit therewith, such as with a ridge 248 of endoscope 200 as discussed above. As will be appreciated, coupling a second end 804 of adapter 800 to a ridge 248 via a press fit or friction fit may not require deformation of the adapter, and thus the adapter 800 may be formed of material with greater rigidity than a material used to form adapter 502. In one non-limiting example embodiment, second end 806, or the entire adapter 800, may be formed of stainless steel.

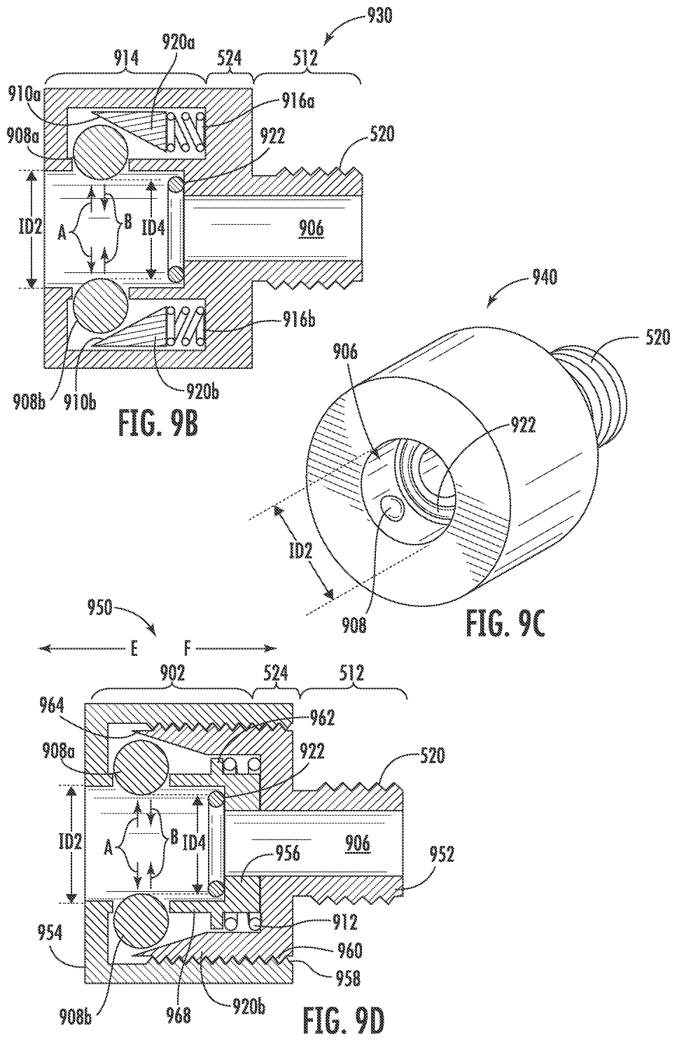

FIGS. 9A-B show cross sectional views of adapters 900, 930, 940 that have quick release connections, or quick disconnect fittings for coupling to the delivery system 100 or endoscope 200. FIG. 9C shows a perspective view of the adapter 900 of FIG. 9A, or adapter 930 of FIG. 9B. FIG. 9D shows a cross sectional view of adapter 950 with a lockable quick release connection, or quick disconnect fitting for coupling to the delivery system 100 or endoscope 200. As will be appreciated, a quick release connection may allow for easy and/or rapid coupling and/or decoupling of the adapter 900, 930, 950 to endoscope 200. For example, adapters 900, 930, 950 may be suited to easily couple to the endoscope 200 channel opening 246 over ridge 248 without requiring deformation of the adapter. For the sake of simplicity, adapters 900, 930, 950 are illustrated each with a middle portion 524 and an end 512, including threaded outer surface 520 for coupling to delivery system 100, as described above. Similar components of adapters 900, 930 will be described together for the sake of additional simplicity. Adapter 950 will be described subsequently, having an alternative lockable configuration.

Adapters 900, 930 may have respective second ends 902, 914 that include quick release connections having one or more spring-loaded bearings 908a-b. Bearings 908a-b may be biased by spring 912 or springs 916a-b so that the bearings extend partially into lumen 906, decreasing the lumen inner diameter ID2 to be a smaller inner diameter “ID4” at the location of their extension into the lumen 906. When ridge 248 of endoscope 200 is introduced into the lumen 906 at the second end 902, 904 of the adapter 900, 930, the ridge 248 presses against the bearings 908a-b and overcomes the spring force of spring 912 or 916a-b, causing the bearings to move radially outward in the direction of arrows “A”, which increases the effective inner diameter of the lumen from ID4 to a diameter sufficient to allow the ridge 248 to pass.

As the bearings 908a-b are moved radially outward, they apply pressure to angled surfaces 910a-b of spring extensions 920a-b. In some embodiments, a separate angled surface may be paired with each bearing. In other embodiments, a single angled surface and/or spring extension may extend circumferentially around the lumen 906 of the adapter. Embodiments are not limited in this context.

Angled surfaces 910a-b translate a force applied by the radially outward movement of the bearings 908a-b to spring 912 or 916a-b. In FIG. 9A, a single spring 912 extends circumferentially about a lumen 906. In FIG. 9B, multiple springs 916a-b may be used. Springs 916a-b may be comparatively smaller than a spring 912, for example, in a cross-sectional area, and therefore may be more easily compressible. However, a single, larger spring 912 may be useful for translating force translated from motion of each bearing into the spring.

When pressure on bearings 908a-b from within lumen 906 is released, for example, as the ridge 248 passes the bearings, the spring 912, 916a-b may expand, causing the angled surfaces 910a-b to translate a radially inward force on the bearings 908a-b (e.g., in the direction of arrows “B”). Accordingly, the bearings 908a-b may partially protrude into the lumen 906, thereby decreasing a corresponding diameter thereof. The ridge 248 may thus be locked within the adapter 900, 930 to lock the adapter to the endoscope 200.

In many embodiments, locking of the ridge 248 within the adapter 900 may cause the ridge 248 to press against an O-ring 922 positioned within lumen 906. In many embodiments, O-ring 922 may be positioned within a portion of lumen 906 with inner diameter ID2. O-ring 922 may form a press fit or seal with ridge 248 so as to prevent fluid flow from channel 250 from leaking through the end 902 of the adapter. O-ring 922 may be formed of silicone, a polymer, or elastomer suitable for forming a fluid-tight seal. It will be recognized that, while not illustrated for the sake of simplicity, any embodiment described herein may comprise an O-ring 922 or similar sealing element.

Referring to FIG. 9D, adapter 950 is illustrated for the sake of simplicity as including various elements described with respect to FIGS. 9A-C, such as lumen 906, bearings 908a-b, spring 912, O-ring 922, arrows A, B, and inner diameters ID2, ID4. It will be understood that embodiments may alternatively or additionally comprise other elements as described herein. For example, spring 912 may be replaced by springs 916a-b.

Adapter 950 can also include multiple additional or alternative structural members such as central coupler 952, outer collar 954, and inner collar 956. At end 966, structural outer collar 954 may be rotatably positioned about central coupler 952. inner collar 956 may be located circumferentially within central coupler 952, together with central coupler 952 defining lumen 906. In many non-limiting examples, inner collar 956 may be biased by spring 912 in the direction of arrow E, for example, via the force of spring 912 on a spring interface 962 of inner collar 956.

In many embodiments, internal threading 958 of outer collar 954 may interact with external threading 960 of central coupler 952 such that rotation of outer collar 954 with respect to central coupler 952 in one direction may cause the outer collar 954 to longitudinally move in a direction of arrow “E,” while central coupler 952 remains stationary. Rotation of outer collar 954 with respect to central coupler 952 in the other direction may cause the outer collar 954 to longitudinally move in a direction of arrow “F,” while central coupler 952 remains stationary. It will be understood that, alternatively, central coupler 952 may be rotated and/or moved with respect to outer collar 954, for example, while outer collar 954 remains stationary.

Movement of outer collar 954 in the direction of arrow F with respect to central coupler 952 will apply a force and corresponding motion in direction the direction of arrows “G” to bearings 908a-b. However, angled surface 964 of central coupler 952 directs bearings 908a-b, as they move in the direction of arrow F, to simultaneously extend radially inward into lumen 906 along arrows B. Accordingly, bearings 908a-b may decrease the lumen inner diameter ID2 to be a smaller inner diameter “ID4” at the location of their extension into the lumen 906. Additionally, movement of bearings 908a-b may overcome the biasing of spring 912 in the direction to cause movement of inner collar 956 in a direction of arrow F with respect to central coupler 952, correspondingly compressing spring 912.

While compressed, spring 912 may bias inner collar 956 to move in the direction of arrow E. However, the interaction of threading 958 and threading 960 may lock the position of central coupler 952 and outer collar 954 with respect to each other, thereby locking the positions of bearings 908a-b and inner collar 956 with respect to central coupler 952 and outer collar 954.

However, movement of outer collar 954 in the direction of arrow E with respect to central coupler 952 will reduce or relieve a force of outer collar 954 on bearings 908a-b in the direction of arrow F. Accordingly, inner collar 956 may move with the biasing of spring 912 to in the direction of arrow E. A projection 968 of central coupler 952 may apply pressure to bearings 908a-b, causing them to move along angled surface 964 in a direction of arrows A and E. Accordingly, bearings 908a-b may increase the lumen inner diameter ID4 to be a larger inner diameter “ID2” at the location of their extension into the lumen 906.

It will be understood that end 966 may be positioned over ridge 248 of endoscope 200 while outer collar 954 is moved along arrow E with respect to central coupler 952 (e.g., while adapter 950 is in an unlocked configuration). Subsequently, outer collar 954 may be rotated with respect to central coupler 952 to cause a movement of outer collar 954 along arrow F and a restriction of a lumen diameter of adapter 950 from ID2 to ID4 distal to the ridge 248. Accordingly, the adapter 950 may secured, or fixed, to the endoscope 200 (e.g., with adapter 950 in a locked configuration). It will be understood that a lockable or fixable coupling of an adapter to an endoscope may improve stability of a coupled system during a procedure, increasing ease of operation for a physician.

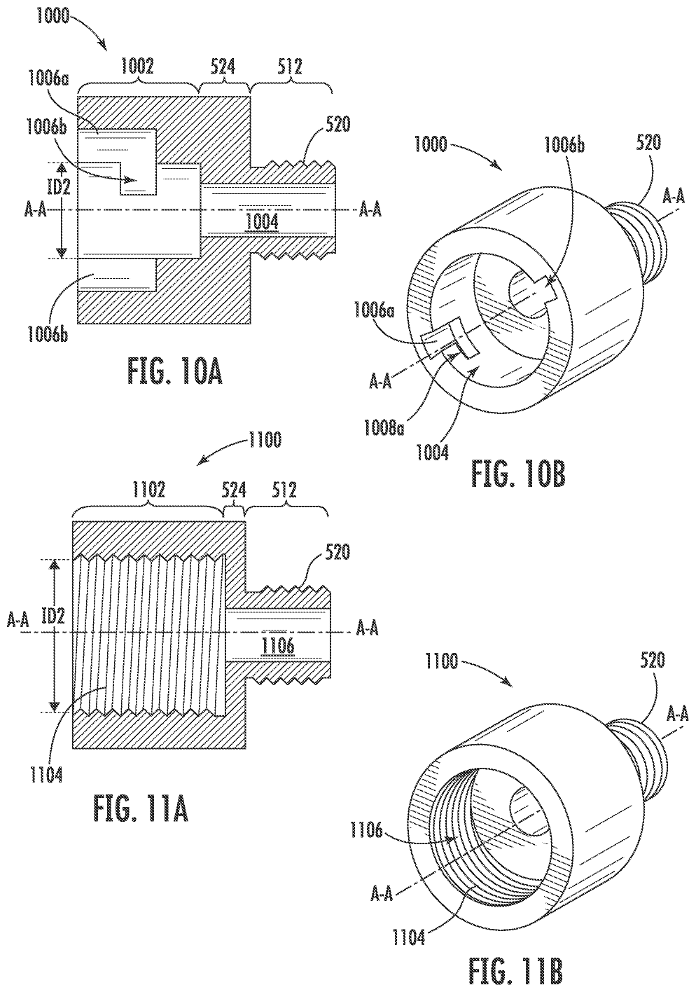

FIG. 10A shows a cross-sectional view of an exemplary adapter 1000 with a partial turn lock connection couplable to medical devices, such as to endoscope channel openings. FIG. 10B shows a perspective view of adapter 1000. For the sake of simplicity, adapter 1000 is illustrated with a middle portion 524 and an end 512, including threaded outer surface 520, as described above. It will be recognized that the adapter 1000 may comprise an end alternative to end 512, for example, end 702 or end 728 as described above.

Adapter 1000 may comprise a first end 1002 with one or more recesses 1006a-b along an interior wall of a lumen 1004 extending therethrough. Recesses 1006a-b may be configured to receive, at the first end 1002 of the adapter, correspondingly sized and positioned projections of a medical device (not shown). As can be seen, the recesses 1006a-b are oriented within the inner sidewalls of the adapter 1000 and are generally parallel to the longitudinal axis A-A of the lumen 1004. A blind recess 1008a-b is associated with each of the recesses 1006a-b. The blind recesses 1008a-b are oriented perpendicular to the longitudinal axis of the lumen 1004 and are configured to lock an associated projection of the medical device therein. Thus, when the projections of the medical device are aligned with the recesses 1006a-b and the medical device is fully inserted into the lumen 1004 corresponding to first end 1002, rotation of the medical device with respect to the adapter 1000 (or vice versa), causes the projections of the medical device to be received within the blind recesses, thereby coupling the medical device to the adapter 1000. While blind recess 1008b is not visible in FIGS. 10A-B, it will be understood that each depression of an adapter with a partial turn-lock connection may be similarly L-shaped, curved, or otherwise shaped. In some embodiments, a partial turn lock connection may be coupled via a quarter rotation, a one-third rotation, a half-rotation, or other partial rotation about axis “A-A,” which extends longitudinally through the center of lumen 1004. In some embodiments, a partial turn lock may comprise a Luer lock fitting.

FIG. 11A shows an exemplary cross-sectional view of an adapter 1100 with a threaded connection suitable for connecting to a medical device, such as endoscope 200s. FIG. 11B shows a perspective view of adapter 1100. Middle portion 524 and end 512, including threaded outer surface 520, as well as axis A-A as described above are included in adapter 1100, but it will be recognized that alternative ends may be used. For example, an adapter 1100 may include an end 702 or end 728 as described above rather than end 512.

Adapter 1100 may comprise a first end 1102, which may include threads 1104 disposed on an inner surface of a lumen 1106 extending through the adapter 1100. The diameter of the lumen 1106 may be, in many examples, larger at the first end 1102 than the second end 512 of the adapter 1100. For example, lumen 1106 may have a diameter of ID2, as discussed above, in a portion corresponding to end 1102. Threads 1104 and threaded outer surface 520 may comprise the same or different thread profiles, for example, having the same or different pitches, depths, and/or directionality.

Threads 1104 can couple with a medical device having corresponding threads on an outer surface thereof. In some embodiments the medical device may include a surface variation such as one or more discrete projections that may be rotated into engagement with the threads 1104 of the adapter 1100, for example, by rotating the adapter about axis A-A with respect to the medical device, by rotating the medical device about axis A-A with respect to the adapter, or both.

As described above, each adapter end may be coupled by a respective action to a medical device, for example, by a rotation or insertion of the medical device into a lumen of the adapter. It will be understood that each device may be respectively decoupled from the medical device by an opposite action. For example, an adapter coupled via a rotation may be decoupled via a rotation in the opposite direction (e.g., adapters 502, 602 with respect to end 512, and/or adapters 1000, 1100 with respect to corresponding ends 1002, 1102). In another example, an adapter via an insertion of the medical device into a lumen thereof or via an insertion of an adapter end into a lumen of the medical device may be decoupled via a respective removal (e.g., adapter 700 with respect to ends 514, 702; adapter 720 with respect to ends 514, 728, adapter 800 with respect to end 804; and/or adapters 900, 930 with respect to ends 902, 914). Additionally, while each of the illustrated adapters is shown with one female fitting and one male fitting, it will be understood that embodiments may comprise any combinations of ends described herein. By way of example, an adapter may comprise a first end 512 and a second end 702, or an adapter may comprise a first end 514 and a second end 914 as described herein (not shown). Accordingly, a variety of medical device connections may be achieved via adapters of the present disclosure.

FIG. 12 shows an alternative delivery device 1200 which may be coupled with one or more adapters described herein. For example, delivery device 1200 may be configured to deliver a needle to a target tissue. A handle 1202 may be used to control a tool deployment through a catheter end 1206, with catheter 1208 extending between the handle and the catheter end. A locking member 1210 may be disposed on the end of handle 1202 and comprise one or more similarities to the locking member 110 as described with respect to FIGS. 1A-B. For example, at least one projection 1220 may be used to grip the locking member 1210 (similar to projections 118, 120), and a lumen 1222 may be configured to couple with an adapter as described herein (for example, an adapter with an end 512). Catheter 1208 may be configured to extend through an adapter (not shown) similarly to shaft 106. A control knob 1212 may be used to extend a tool (not shown), such as a needle, from the catheter end 1206, for example, via an iterative predetermined distance according to a corresponding rotation of the control knob 1212. A locking knob 1214 may be used to secure a position of the tool with respect to the delivery device 1200. One or more echogenic, radiopaque, colored, otherwise imageable markings, or any combination thereof may be placed along a sliding portion 1216 extending between control knob 1212 and locking knob 1214, a catheter 1208, or a catheter end 1206 which may aid in determining a positioning of the delivery system. It will be appreciated that coupling a delivery device 1200 with an adapter as described herein may allow the delivery system to be coupled and used during a procedure in coordination with a variety of other medical devices, such as endoscope 200.

Components may be coupled and/or decoupled prior to or during a procedure according to a physician's need and/or convenience. It will also be understood that adapters may comprise a variety of dimensions corresponding to dimensions of medical devices desired to be used. For example, a diameter D of a catheter may be in a range of 5-12 Fr. For a catheter with diameter D in the range of 5-12 Fr, a corresponding inner diameter ID1 of an adapter may be in a range of 5-12 Fr, or in a range of 5-13 Fr, for example, being substantially the same or slightly larger than D. In various non-limiting examples, a diameter D of a catheter may be in a range of 6-11 Fr, for example, in a range of 6-8 Fr or in a range of 9-11 Fr. Threading as described herein may be right-handed or left-handed. In some embodiments (not shown), threading may be configured to couple to threading of multiple pitches and/or sizes, thereby increasing an adaptability of the adapter to a greater array of medical devices.

In various embodiments, an entire longitudinal length of an adapter may be in the range of 2-3 cm, or in the range of 1-4 cm, although an entire longitudinal length of an adapter may be in the range of 1-15 cm. In many embodiments, the summation of lengths of a middle portion and a first end of an adapter, such as respectively marked L1 and L2 in FIG. 3A, may be in the range of 1-2 cm. However, the summation of lengths of a middle portion and a first end of an adapter may have a length in the range of 1-15 cm. Embodiments are not limited in this context.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

What is claimed is:

1. A system, comprising:
a locking member of a medical device delivery system; and
a coupling device,
a first end of the coupling device comprising a portion with a threaded outer surface configured to couple to the locking member, the first end further comprising a lumen extending therethrough;
a second end of the coupling device comprising a lumen axially continuous with the lumen of the first end, wherein the second end is configured to couple to an outlet of an endoscope channel.

2. The system of claim 1, wherein the first end of the coupling device comprises a Luer lock fitting.

3. The system of claim 1, wherein the lumen at the second end of the coupling device comprises a first portion with a first inner diameter and a second portion with a second inner diameter, the second inner diameter larger than the first inner diameter, wherein the second portion is configured to fit over a ridge of the outlet of the endoscope channel.

4. The system of claim 1, further comprising a cap coupled to the first end of the coupling device.

5. The system of claim 1, the coupling device further comprising a middle portion between the first end and the second end, wherein an external surface of the middle portion comprises a surface variation.

6. The system of claim 1, the coupling device further configured to couple to the locking member to create a fluid-tight fit.

7. The coupling device of claim 1, wherein the lumen of the coupling device comprises a unidirectional valve.

8. The system of claim 1, wherein an inner diameter of at least part of the lumen of the coupling device is included in a range of 5-12 Fr.

9. The system of claim 1, wherein the first end of the coupling device, when coupled to the locking member, is configured to prevent free rotation of the coupling device with respect to the locking member,
wherein the second end of the coupling device, when coupled to a channel of an endoscope, is configured to prevent free rotation of the coupling device with respect to the channel,
or both.

10. The system of claim 1, wherein the lumen of the second end of the coupling device is configured to receive an outlet of an endoscope channel in a fluid-tight fit.

11. The system of claim 1, wherein, when the first end of the coupling device is coupled to the locking member and the second end of the coupling device is coupled to an outlet of an endoscope channel, the lumen of the first end is configured to pass a delivery sheath of the medical device delivery system into the endoscope channel.

12. The coupling device of claim 1, wherein the second end of the coupling device is configured to couple to an outlet of an endoscope channel via a press fit, a turn lock connection, quick disconnect fitting, or a clip fit.

13. A system for connecting a medical device to an endoscope, comprising:
a locking member of the medical device; and
a coupling device, wherein the coupling device comprises:
a first end comprising threading along an outer surface thereof configured to be coupled with a locking member, wherein the locking member is disposed around a sheath of the medical device;
a second end configured to be coupled with an outlet of a channel of an endoscope; and
a lumen extending longitudinally along a full length of the first end and the second end,
wherein the second end is configured to couple to the outlet via insertion of the outlet into the lumen.

14. The system of claim 13, the coupling device further configured to couple to the locking member to create a fluid-tight fit, to the outlet to create a fluid-tight fit, or both.

15. The system of claim 13, wherein the lumen of the coupling device comprises a unidirectional valve.

16. The system of claim 13, wherein an inner diameter of at least part of the lumen of the coupling device is included in a range of 5-12 Fr.

17. The system of claim 13, wherein the first end of the coupling device, when coupled to the locking member, is configured to prevent free rotation of the coupling device with respect to the locking member,
wherein the second end of the coupling device, when coupled to a channel of an endoscope, is configured to prevent free rotation of the coupling device with respect to the channel,
or both.

18. The system of claim 13, wherein, when the first end of the coupling device is coupled to the locking member and the second end of the coupling device is coupled to an outlet of an endoscope channel, the lumen of the first end of the coupling device is configured to pass a delivery sheath of the medical device into the endoscope channel.

19. The system of claim 13, wherein the second end of the coupling device is configured to couple to an outlet of an endoscope channel via a press fit, a turn lock connection, quick disconnect fitting, or a clip fit.

20. A system for coupling a delivery system to an endoscope, comprising:
a locking member of a delivery system; and
an adapter, wherein the adapter includes:
a first end configured to, when inserted into a lumen of a locking member of the delivery system, secure the adapter to the locking member; and
a second end configured to clip over a ridge of a channel opening of the endoscope,
wherein, when the second end is clipped over the ridge of the channel opening, the first end forms a lumen providing access through the adapter to the channel opening.
